(19) United States
(12) Patent Application Publication
(10) Pub. No.: US 2023/0363721 A1
(43) Pub. Date: Nov. 16, 2023

(54) APPARATUSES AND SYSTEMS FOR ARTIFACT REDUCTION IN ELECTRODERMAL ACTIVITY

(71) Applicant: University of Connecticut, Farmington, CT (US)

(72) Inventors: Ki Chon, Mansfield, CT (US); Md Billal Hossain, Willington, CT (US); Hugo Posada-Quintero, Vernon, CT (US)

(21) Appl. No.: 18/313,734

(22) Filed: May 8, 2023

Related U.S. Application Data
(60) Provisional application No. 63/340,272, filed on May 10, 2022.

(51) Int. Cl. A61B 5/00

(52) U.S. Cl. CPC A61B 5/7282; A61B 5/7203; A61B 5/7267

(57) ABSTRACT

Methods, systems, non-transitory computer-readable media, and apparatuses are described for predicting a health condition of a subject. An apparatus may be configured to receive a physiological signal associated with the subject. The physiological signal may include artifacts. A modified physiological signal may be generated based on an application of a machine learning model to the physiological signal. The modified physiological signal may include the physiological signal with a reduction of the artifacts. A physiological measurement may be determined based on the modified physiological signal. The health condition may be determined based on a change in the physiological measurement satisfying a threshold. The apparatus may cause an output of an indication associated with the health condition.

### CROSS REFERENCE TO RELATED PATENT APPLICATION

This application claims priority to U.S. Provisional Application No. 63/340,272, filed May 10, 2022, which is herein incorporated by reference in its entirety.

### FEDERALLY SPONSORED RESEARCH

This invention was made with government support under N00014-21-1-2255, awarded by the Office of Naval Research. The government has certain rights in the invention.

### BACKGROUND

This application generally relates to reducing artifacts in an electrodermal activity (EDA) signal and determining a health condition of a subject based on the EDA signal. Decompression sickness (DCS) in divers is caused by the formation of nitrogen bubbles in the blood or body tissues when there is sudden reduction in ambient pressure during the ascent phase of a dive. During a prolonged hyperbaric exposure, intake gases dissolve in the blood, then emerge from the solution as bubbles in body tissues during decompression. Symptoms of DCS may range from mild (e.g., skin itching, muscle pain, nausea, and tingling) to serious including neurological dysfunctions, spinal cord injury, cardiopulmonary collapse and disseminated intravascular coagulation. An effective way to mitigate DCS in certain situations is hyperbaric oxygen (HBO₂) pre-breathing under water. Unfortunately, breathing HBO₂ creates a risk for divers to develop central nervous system oxygen toxicity (CNS-OT). The symptoms of CNS-OT include headache, diaphoresis, nausea, tinnitus, lip twitching, tingling of the limbs, or even more serious symptoms such as seizure or loss of consciousness. Although, oxygen-induced seizures are not life-threatening in the controlled, dry environment of a hyperbaric chamber, losing consciousness or convulsing under water could result in the dislodgement of the diver's air supply from his or her mouth, and likely lead to drowning.

Studies involving animal and human models breathing HBO₂ in a pressurized chamber have shown consistent seizure activities due to CNS-OT induced by HBO₂. Physiologically, augmented seizure activity is associated with elevated sympathetic activity. Therefore, a sensitive measure of sympathetic activity can be used as a surrogate physiomarker of seizure detection and more importantly seizure prediction. For example, electrodermal activity (EDA) has been used as a measure of sympathetic activity, especially due to the use of wearable technologies that may be used to monitor EDA. Conventional methods of analyzing EDA have been confined to the time domain, decomposing the signal into tonic and phasic EDA. The tonic EDA is typically quantified using the skin conductance level (SCL), the mean value of the tonic component. The phasic EDA is comprised of the skin conductance responses (SCRs). The SCRs are the rapid transient events contained in the EDA signal. However, it has been shown that there is a low reproducibility of these time-domain measures.

Generally, the term “electrodermal activity (EDA)” refers to a property of the human body that causes continuous variation in the electrical characteristics of the skin. Historically, EDA has also been known as skin conductance, galvanic skin response (GSR), electrodermal response (EDR), psychogalvanic reflex (PGR), skin conductance response (SCR), sympathetic skin response (SSR) and skin conductance level (SCL). The long history of research into the active and passive electrical properties of the skin by a variety of disciplines has resulted in an excess of names, now standardized to electrodermal activity (EDA). The traditional theory of EDA holds that skin resistance varies with the state of sweat glands in the skin. Sweating is controlled by the sympathetic nervous system, and skin conductance is an indication of psychological or physiological arousal. If the sympathetic branch of the autonomic nervous system is highly aroused, then sweat gland activity also increases, which in turn increases skin conductance. In this way, skin conductance can be a measure of emotional and sympathetic responses. More recent research and additional phenomena (resistance, potential, impedance, electrochemical skin conductance, and admittance, sometimes responsive and sometimes apparently spontaneous) suggest that EDA is complex and can be difficult to measure accurately.

For example, accurate measurement of EDA data is often hampered by motion artifacts, especially EDA data obtained via wearable technologies. These artifacts can be severe enough to cause the data to be unreliable, or corrupted, and thus, unusable. One conventional method of removing motion artifacts from the EDA data involves detecting and discarding corrupted data segments. However, this conventional method can be quite costly as the amount of usable data can be significantly reduced. Another conventional method involves the use of an unsupervised scheme using a one-class support vector machine and k-nearest neighbor distance to automatically detect motion artifacts. However, this conventional method has yet to be successfully applied to EDA data that have more dynamics. Other conventional methods involve the use of exponential smoothing and low pass filtering to remove motion artifacts from the EDA data. However, these conventional methods are non-adaptive, fail to remove artifacts with high intensity, and are subject to distorting the skin conductance response especially in portions of the data segments without artifacts. Conventional autoencoder methods involve an unsupervised artificial neural network to learn efficient coding of unlabeled data, which is then validated and refined to reproduce the original input. However, if the model is sufficiently large with many parameters to tune, it may learn an identity function so that the output is always equal to the input, and thus, the network may not create a useful representation of the data.

What are needed are techniques for reducing motion artifacts in an electrodermal activity (EDA) signal and determining a health condition of a subject based on the EDA signal. Preferably, the techniques result in rapid delivery of accurate results. Techniques should be efficient computing wise, and therefore available for implementation in low computing power systems, such as in wearable technology.

### SUMMARY

It is understood that both the following general description and the following detailed description are exemplary and explanatory only and are not restrictive. Methods, systems, and apparatuses for improved artifact reduction in electrodermal activity (EDA) and for improved health condition determinations of a subject based on EDA are described.

In some embodiments, an apparatus for predicting a health condition of a subject is provided. The apparatus may include one or more processors and a memory storing processor-executable instructions that, when executed by the one or more processors. The apparatus may be configured to receive a physiological signal associated with the subject. The physiological signal may include artifacts. A modified physiological signal may be generated based on an application of a machine learning model to the physiological signal. The modified physiological signal may include the physiological signal with a reduction of the artifacts. A physiological measurement may be determined based on the modified physiological signal. The health condition may be determined based on a change in the physiological measurement satisfying a threshold. The apparatus may cause an output of an indication associated with the health condition.

In some embodiments, one or more non-transitory computer-readable media storing processor-executable instructions for predicting a health condition of a subject are provided. The processor-executable instructions may cause the at least one processor to receive a physiological signal associated with the subject by a computing device when executed by at least one processor. The physiological signal may include artifacts. A modified physiological signal may be generated based on an application of a machine learning model to the physiological signal. The modified physiological signal may include the physiological signal with a reduction of the artifacts. A physiological measurement may be determined based on the modified physiological signal. The health condition may be determined based on a change in the physiological measurement satisfying a threshold. The processor-executable instructions may cause an output of an indication associated with the health condition.

In an embodiment, are systems for predicting a health condition of a subject associated with prolonged exposure to hyperbaric oxygen (HBO₂). The system may include a senor affixed to a surface of skin of the subject, a display configured to output an interface to the subject, and a computing device in communication with the sensor and the display. The sensor may be configured to determine a physiological signal associated with the subject based on measuring a conductance associated with the surface of skin of the subject. The display may be configured to output an interface to the subject. The computing device may be configured to receive the physiological signal from the sensor. The physiological signal may include artifacts. The physiological signal may be provided to a machine learning model. A modified physiological signal may be generated based on the machine learning model. The modified physiological signal may include the physiological signal with a reduction of the artifacts. A physiological measurement may be determined based on the modified physiological signal. A change in the physiological measurement may be determined to satisfy a threshold. The change may be caused by stress based on breathing performed by the subject during prolonged exposure to HBO₂. The health condition may be determined based on the change in the physiological measurement satisfying the threshold. The computing device may be configured to cause an output of an indication of the health condition via the interface to the subject.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings serve to explain the principles of the methods, apparatuses, and systems described herein:

FIG. 1 shows an example system;

FIG. 2 shows an example system environment;

FIG. 3 shows an example machine learning system;

FIG. 4 shows a flowchart of an example training method;

FIGS. 5A-5B show example physiological measurements;

FIG. 6 shows a flowchart of an example method; and

FIG. 7 shows a block diagram of a computing device for implementing the example methods.

### DETAILED DESCRIPTION

Methods, systems, and apparatuses are described for reducing artifacts in an electrodermal activity (EDA) signal and for determining a health condition based on the EDA signal. The artifacts may be caused by one or more of motion artifacts arising from variable conduction signals as a subject moves or electronic noise arising from radiofrequency and magnetic interference. A deep convolutional autoencoder network (DCAE) may be applied to an EDA signal associated with a subject to reduce artifacts in the EDA signal due to movements performed by the subject and produce a modified EDA signal. The DCAE may be trained based on one or more training data sets associated with one or more EDA signals associated with one or more subjects. The modified EDA signal may be used to determine a time-invariant and/or a time-variant spectral analysis of the EDA signal (TVSymp). A health condition may be determined based on the TVSymp. For example, the health condition may be determined based on a change in the TVSymp satisfying a threshold. The change may be caused by stress experienced by the subject based on breathing performed by the subject during prolonged exposure to hyperbaric oxygen (HBO₂). The health condition may include one or more of a risk of seizure, a risk of central nervous system oxygen toxicity (CNS-OT), or symptoms of CNS-OT. The health condition may be output via an interface to the subject.

Generally, as discussed herein, the term “physiological signal” refers to signals obtained from a subject such as electrodermal activity (EDA) and other related or associated quantities.

Generally, as discussed herein, the term “artifact” refers to spurious contributions to meaningful data that may arise from, for example, motion noise and/or electronic noise. Motion artifacts may arise from, for example, variable conduction of signals as the subject moves about. Electronic noise may arise from, for example, radiofrequency and magnetic interference.

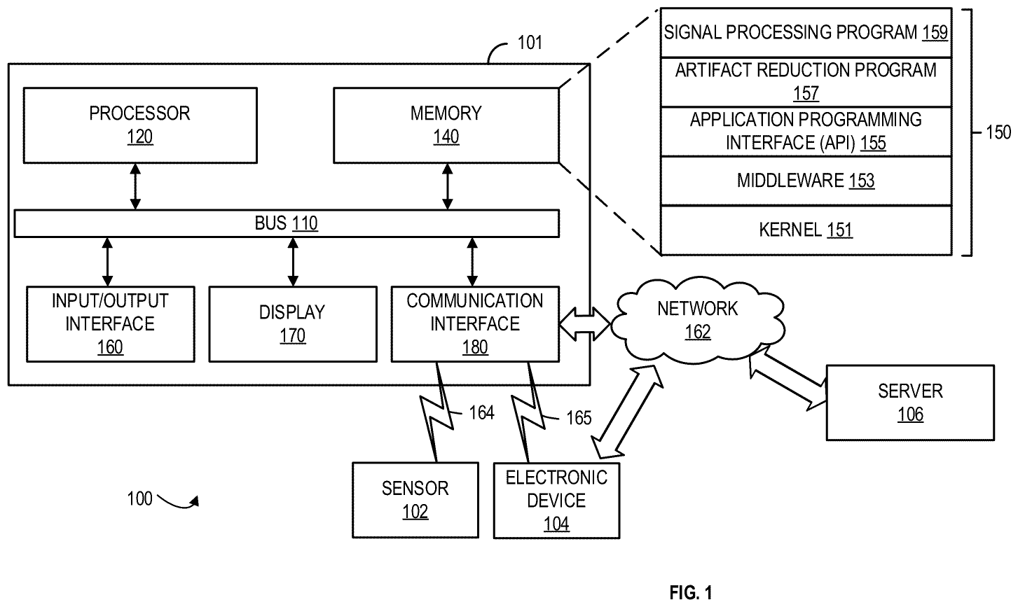

FIG. 1 shows an example system 100 reducing artifacts in an EDA signal associated with a subject and determining a health condition of the subject based on the EDA signal. For example, a time-invariant and/or a time-variant spectral analysis of the EDA signal (TVSymp) may be determined based on the EDA signal. A change in the TVSymp may be used to determine a health condition (e.g., a risk of seizure, a risk of CNS-OT, or symptoms of CNS-OT) associated the subject during prolonged exposure to HBO₂. The system 100 may include a device 101 that may be configured to use one or more methods for reducing artifacts in the EDA signal to facilitate the determination of the health condition. The device may be in communication with one or more sensors 102, one or more electronic devices 104, and one or more servers 106. The device may include a bus 110, one or more processors 120, a memory 140, an input/output interface 160, a display 170, and a communication interface 180. In a certain examples, the device 101 may omit at least one of the aforementioned elements or may additionally include other elements. The device 101 may include a wearable device, a smart watch, tablet computer, a laptop computer, a desktop computer, and the like.

The bus 110 may include a circuit for connecting the bus 110, the one or more processors 120, the memory 140, the input/output interface 160, the display 170, and/or the communication interface 180 to each other and for delivering communication (e.g., a control message and/or data) between the bus 110, the one or more processors 120, the memory 140, the input/output interface 160, the display 170, and/or the communication interface 180.

The one or more processors 120 may include one or more of a Central Processing Unit (CPU), an Application Processor (AP), or a Communication Processor (CP). The one or more processors 120 may control, for example, at least one of the bus 110, the memory 140, the input/output interface 160, the display 170, and/or the communication interface 180 of the device 101 and/or may execute an arithmetic operation or data processing for communication. For example, the one or more processors 120 may drive (e.g., cause) the display 170 and/or a speaker to issue visual and/or audio instructions or signals, respectively to an operator (e.g., subject) of the device 101, such as an indication of a health condition of the subject. For example, the health condition may include one or more of a risk of seizure, a risk of CNS-OT, or symptoms of CNS-OT. The processing (or controlling) operation of the one or more processors 120 according to various embodiments is described in detail with reference to the following drawings.

The processor-executable instructions executed by the one or more processor 120 may be stored and/or maintained by the memory 140. The memory 140 may include a volatile and/or non-volatile memory. The memory 140 may include random-access memory (RAM), flash memory, solid state or inertial disks, or any combination thereof. The memory 140 may store, for example, a command or data related to at least one of the bus 110, the one or more processors 120, the memory 140, the input/output interface 160, the display 170, and/or the communication interface 180 of the device 101. According to various examples, the memory 140 may store software and/or a program 150. For example, the program 150 may include a kernel 151, a middleware 153, an Application Programming Interface (API) 155, an artifact reduction program 157, and/or a signal processing program 159, or the like, configured for controlling one or more functions of the device 101 and/or an external device (e.g., sensor 102 or electronic device 104). At least one part of the kernel 151, middleware 153, or API 155 may be referred to as an Operating System (OS). The memory 140 may include a computer-readable recording medium (e.g., a non-transitory computer-readable medium) having a program recorded therein to perform the methods according to various embodiments by the one or more processors 120.

The kernel 151 may control or manage, for example, system resources (e.g., the bus 110, the processor 120, the memory 140, etc.) used to execute an operation or function implemented in other programs (e.g., the middleware 153, the API 155, the artifact reduction program 157, or the signal processing program 159). Further, the kernel 151 may provide an interface capable of controlling or managing the system resources by accessing individual elements of the device 101 in the middleware 153, the API 155, the artifact reduction program 157, or the signal processing program 159.

The middleware 153 may perform, for example, a mediation role, so that the API 155, the artifact reduction program 157, and/or the signal processing program 159 can communicate with the kernel 151 to exchange data. Further, the middleware 153 may handle one or more task requests received from the artifact reduction program 157 and/or the signal processing program 159 according to a priority. For example, the middleware 153 may assign a priority of using the system resources (e.g., the bus 110, the one or more processors 120, or the memory 140) of the device 101 to at least one of the artifact reduction program 157 and/or the signal processing program 159. For example, the middleware 153 may process the one or more task requests according to the priority assigned to at least one of the application programs, and thus, may perform scheduling or load balancing on the one or more task requests.

The API 155 may include at least one interface or function (e.g., instruction), for example, for file control, window control, video processing, and/or character control, as an interface capable of controlling a function provided by the artifact reduction program 157 and/or the signal processing program 159 in the kernel 151 or the middleware 153.

In an example, the artifact reduction program 157 and the signal processing program 159 may be independent of each other or integrally combined, in whole or in part.

The artifact reduction program 157 may include logic (e.g., hardware, software, firmware, etc.) that may be implemented to perform the reduction of artifacts in a physiological signal of a subject received from the sensors 102. For example, the physiological signal may include an electrodermal activity (EDA) signal. For example, the artifact reduction program 157 may be used to reduce the artifacts in the EDA signal to enable the signal processing program 159 to process a clean EDA signal (e.g., with a reduction of artifacts) to determine a health condition of the subject. For example, the artifact reduction program 157 may provide a physiological signal received from the sensors 102 to a machine learning model. The machine learning model may include a deep convolutional autoencoder (DCAE) network. The deep convolutional autoencoder network may generate a modified physiological signal. The modified physiological signal may include the physiological signal with a reduction of the artifacts. The machine learning model may be trained based on one or more training data sets. As an example, a training data set may include physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions. As an example, a training data set may include physiological signals associated with a prevalence of artifacts. As an example, a training data set may include physiological signals associated with one or more subjects experiencing central nervous system oxygen toxicity (CNS-OT) conditions. As an example, a training data set may include physiological signals associated with a first sub data set that may include physiological signals associated with a reduction of artifacts collected from both hands of one or more subjects and a second sub data set that may include physiological signals associated with a prevalence of artifacts collected from a first hand of one or more subjects and physiological signals associated with a reduction of artifacts collected from a second hand of one or more subjects.

The signal processing program 159 may include logic (e.g., hardware, software, firmware, etc.) that may determine a health condition of the subject based on the modified physiological signal. The health condition may include one or more of a risk of seizure, a risk of CNS-OT, or symptoms of CNS-OT. For example, the signal processing program 159 may be configured to determine a physiological measurement based on the modified physiological signal. The physiological measurement may include a time-invariant and/or a time-variant spectral analysis of the EDA signal (TVSymp). The signal processing program 159 may determine the health condition based on a change in the physiological measurement (e.g., TVSymp). For example, the change may be caused by stress experience by the subject based on breathing performed by the subject during prolonged exposure to hyperbaric oxygen (HBO₂). For example, the health condition may be determined based on the change in the physiological measurement (e.g., TVSymp) satisfying a threshold. The change may include an increase in the phasic components of the EDA. The increase in the phasic components of the EDA may cause the physiological measurement (e.g., TVSymp) to increase. In an example, the signal processing program 159 may first determine that the physiological measurement (e.g., TVSymp) satisfies a condition. For example, the condition may include whether the change in the physiological measurement (e.g., TVSymp) is based on an autonomic induced elevation of a phasic component of the EDA signal or a non-autonomic induced elevation of a phasic component of the EDA signal. The signal processing program 159 may determine that a change in the physiological measurement (e.g., TVSymp) satisfies a threshold, and thus, indicating a health condition of the subject, based on the physiological measurement (e.g., TVSymp) satisfying the condition. In an example, the determination of whether the physiological measurement (e.g., TVSymp) satisfies the condition may be based on an application of a machine learning model to the physiological measurement (e.g., TVSymp). The signal processing program 159 may cause the device 101 to output an indication associated with the health condition to the display 170 to be displayed to the user (e.g., subject). In an example, the signal processing program 159 may cause the device 101 to output the indication to a server 106. The server 106 may be configured to contact emergency services based on the indication. In an example, the signal processing program 159 may cause the device 101 to output the physiological signal and/or the physiological measurement to the server 106 to be saved in a database (e.g., as one or more files). The database (e.g., server 106) may group the physiological signal and/or the physiological measurement based on the user (e.g., subject).

The input/output interface 160 may include an interface for delivering an instruction or data input from a subject (e.g., an operator of the device 101) or a different external device to the different elements of the device 101. Further, the input/output interface 160 may output an instruction or data received from one or more elements of the device 101 to one or more external devices.

The display 170 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. In an example, the display 170 may include a head mounted display device or a heads up display device. For example, divers may utilize dive masks while diving, especially when deep sea diving. The display 170 may be configured into the dive mask as a heads up display for displaying an interface via a window of the dive mask. The display 170 may display, for example, a variety of contents (e.g., text, image, video, icons, symbols, etc.) to the user (e.g., subject). For example, the display 170 may be configured to output an indication associated with the health condition to the user (e.g., subject). For example, the indication may include an alert notifying the user (e.g., subject) of a possible impending seizure or other health conditions such as a risk of CNS-OT, or that the user (e.g., subject) may be experiencing symptoms of CNS-OT.

The communication interface 180 may establish, for example, communication between the device 101 and one or more external devices (e.g., sensors 102, an electronic device 104, or a server 106). For example, the communication interface 180 may communicate with the one or more external devices (e.g., the server 106 and/or the electronic device 104) by being connected to a network 162 through wireless communication or wired communication. The network 162 may include, for example, at least one of a telecommunications network, a computer network (e.g., LAN or WAN), the Internet, and/or a telephone network.

The communication interface 180 may be configured to communicate with the one or more external devices (e.g., sensors 102, or electronic device 104) via a wired communication interface 164, 165 or a wireless communication interface 164, 165. In an example, the wired communication may include, for example, at least one of Universal Serial Bus (USB), High Definition Multimedia Interface (HDMI), Recommended Standard-232 (RS-232), power-line communication, Plain Old Telephone Service (POTS), and the like. In an example, as a cellular communication protocol, the wireless communication interface 164, 165 may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. In an example, the wireless communication interface 164, 165 may be configured to use a near-distance communication 164, 165. The near-distance communication interface 164, 165 may include for example, at least one of Wireless Fidelity (WiFi), Bluetooth, Bluetooth Low Energy (BLE), Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. In an example, the communication interface 180 may include or be communicably coupled to a transmitter, receiver and/or transceiver for communication with the external devices (e.g., sensors 102, or electronic device 104).

One or more sensors 102 may be in communication with the device via the communication interface connection 164. In an example, the device 101 and the sensors 102 may be configured as two separate devices or may be integrally combined into a signal device. As a single device the sensors 102 may be connected to the bus 110. For example, the sensors 102 may communicate with the one or more processors 120, the memory 140, the input/output interface 160, the display 170, and/or the communication interface 180 via the bus 110. The one or more sensors 102 may include one or more sensors capable of measuring electrodermal activity (EDA) of a user (e.g., subject). For example, the sensors 102 may be configured to measure the conductance associated with a surface of an area of skin of the user (e.g., subject). In an example, the sensors 102 may be integrated into a smart watch (e.g., device 101). In an example, the sensors 102 may be configured to output EDA data to the device 101 for further processing.

An electronic device 104 may be in communication with the device 101 via the communication interface connection 165. The electronic device 104 may include, for example, a head mounted display, a heads up display device, a laptop computer, a mobile phone, a smart phone, a tablet computer, and the like. In an example, the device 101 may be configured to receive the EDA data from the sensors 102 and output the EDA data to the electronic device 104 for further processing. For example, the electronic device 104 may be configured to generate the modified EDA signal based on the EDA data received from the device 101. The electronic device 104 may be configured to determine the health condition of the user (e.g., subject) based on the modified EDA signal. In an example, as a head mounted display or a heads up display device, the electronic device 104 may be configured to receive the physiological measurement (e.g., TVSymp) from the device 101 and output (e.g., display) the physiological measurement (e.g., TVSymp) or the indication associated with the health condition to the user (e.g., subject). For example, the electronic device 104 may output an alert, or notification, notifying the user of a possible impending seizure or other health conditions such as a risk of CNS-OT, or that the user (e.g., subject) may be experiencing symptoms of CNS-OT.

The server 106 may include a group of one or more servers. For example, all or some of the operations executed by the device 101 may be executed in a different one or a plurality of electronic devices (e.g., the device 101, the electronic device 104, and/or the server 106). In an example, if the device 101 needs to perform a certain function or service either automatically or based on a request, the device 101 may request at least some parts of functions related thereto alternatively or additionally to a different electronic device (e.g., the electronic device 104 and/or the server 106) instead of executing the function or the service autonomously. The different electronic devices (e.g., the electronic device 104, or the server 106) may execute the requested function or additional function, and may deliver a result thereof to the device 101. The device 101 may provide the requested function or service either directly or by additionally processing the received result. For example, a cloud computing, distributed computing, or client-server computing technique may be used. In an example, the server 106 may receive the indication associated with the health condition of the user (e.g., subject) and contact emergencies services based on the indication. In an example, the server may receive the physiological signal and/or the physiological measurement and store the physiological signal and/or the physiological measurement in one or more databases. The server 106 may group (e.g., in the one or more databases) the physiological signal and/or the physiological measurement based on the user (e.g., subject).

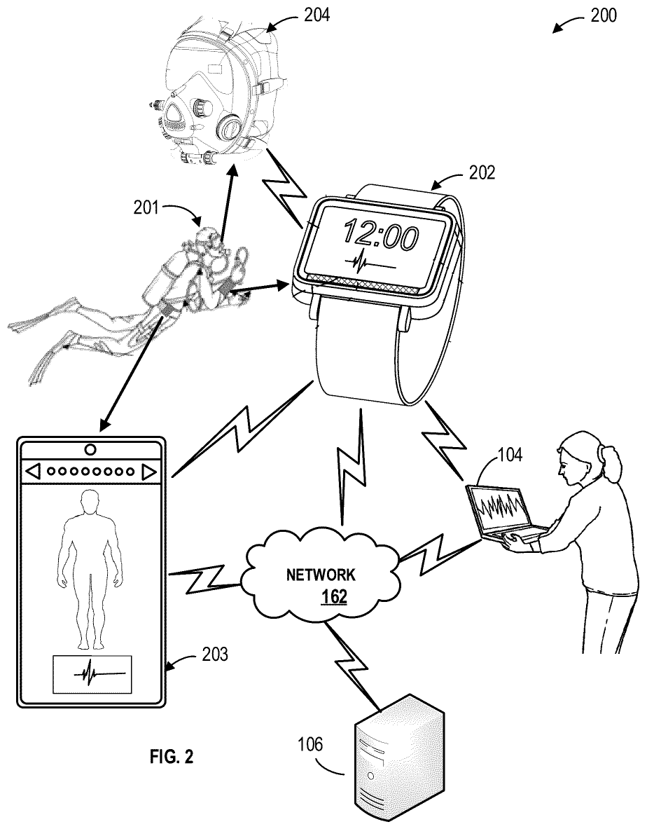

FIG. 2 shows an example system environment 200. The system may include a device 202 (e.g., smart watch, other wearable devices, and the like), a computing device 203 (e.g., smart phone, mobile phone, and the like), a display device 204 (e.g., head mounted display, heads up display device, and the like), the electronic device 104 (e.g., laptop computer, tablet computer, and the like), and/or a server 106. The device 202 may include the sensors 102. The sensors 102 may be affixed to the subject 201 such that the sensors 102 are in contact with a surface of skin of the subject 201. The sensors 102 may measure EDA of the subject and output the EDA to the computing device 203 for further processing. As shown in FIG. 2, the device 202 may be a fully integrated device configured to process the EDA measured by the sensors 102 and output an indication of a health condition based on the EDA to the subject 201. The health condition may include one or more of a risk of seizure, a risk of CNS-OT, or symptoms of CNS-OT. In an example, the computing device 203 may be configured to receive the EDA data from the device 202 and output the indication to the subject 201 based on the EDA data. In an example, the display device 204 may receive the indication from the device 202, and/or the computing device 203, and display the indication to the subject 201. In an example, the device 202 and/or the computing device 203 may be in communication with the electronic device 204 via network 162. For example the device 202 and/or the computing device 203 may output the EDA signal to the electronic device 104. For example, a separate user may use the electronic device 104 to monitor the EDA signal of the subject 201. The electronic device 104 may be configured to determine the health condition based on the EDA signal and output an indication of the health condition to the separate user via the electronic device 104. In an example, the device 202 and/or the computing device 203 may output the indication of the health condition to the electronic device 104 via network 162. The electronic device 104 may then output the received indication to the separate user. In an example, the device 202 and/or the computing device 203 may be in communication with the server via network 162. For example, the device 202 and/or the computing device 203 may output the EDA signal to the server 106. The server 106 may be configured to determine the health condition based on the EDA signal and contact emergency services based on the health condition. In an example, the device 202 and/or the computing device 203 may output the indication of the health condition to the server 106 via network 162. The server 106 may be configured to contact emergency services based on the received indication.

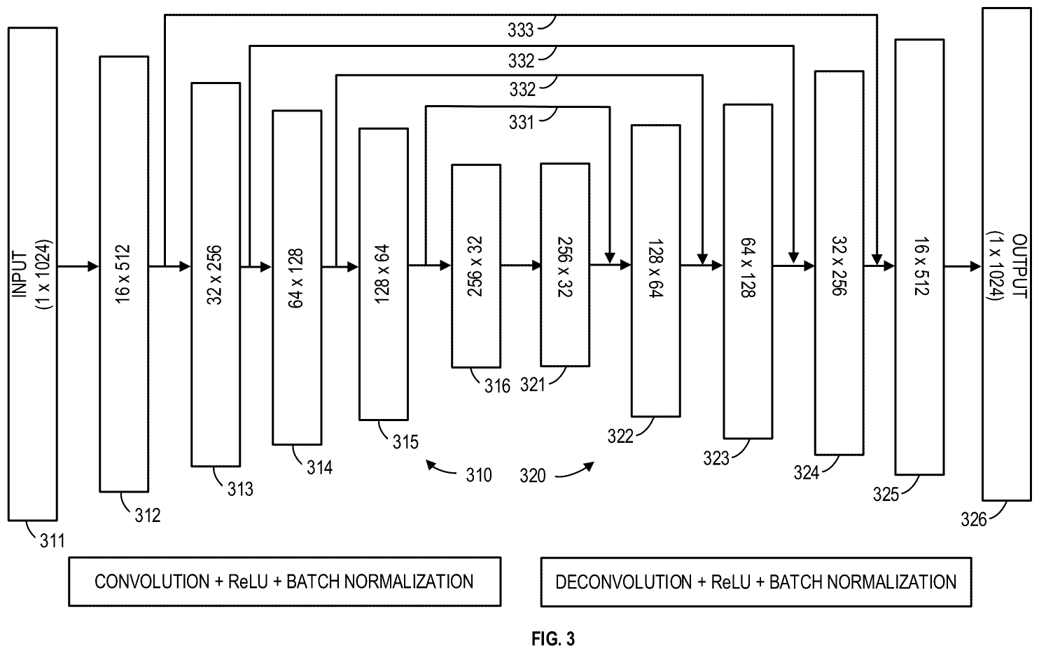

FIG. 3 shows an example machine learning system 300. For example, the machine learning system 300 may include a machine learning model such as a deep convolutional autoencoder (DCAE) network 300. The DCAE network 300 may be configured to include an encoder network 310 and a decoder network 320. The DCAE network 300 may be configured to encode data received at an input layer of the encoder network 310 through layers 312, 313, 314, and 315 to an output layer 316 of the encoder network 310. For example, input layer 311 may include a 1×1024 layer. Features may be upscaled through layers 321, 322, 323, 324, 325, to output layer 326 of decoder network 320. As an example, the decoder network 320 may mirror the encoder network 310 having the same quantity and size layers 321, 322, 323, 324, 325, 326 in reverse order. The DCAE network 300 may be trained to receive an EDA signal associated with a subject that may contain artifacts and output a modified EDA signal with reduced artifacts. For example, the artifacts may be caused by one or more of motion noise or electronic noise. For example, motion artifacts may be caused by variable conduction of signals as the subject moves about and electronic noise may be caused by from radiofrequency and magnetic interference.

As an example, the DCAE network 300 may include one or more convolutional blocks. Each convolutional block may include a 1D convolution layer (with stride=2), ReLU activation function, and batch normalization. Each convolutional block may reduce the input dimension by half. As shown in FIG. 3, the dimension of the data at every stage may include the number of features (e.g., channels)×time stamps. Convolution with stride may be used to reduce the dimension at each layer. The decoder network may be symmetric to the encoder network. The decoder network may deconvolve the encoded vector through different blocks. Each decoder block (except the last block which only consists of a transposed convolutional layer) may include a transposed convolution operation with stride 2 which may up sample the input sequence by a factor of 2, a ReLU activation function, and batch normalization. The DCAE network 300 may include skip connections (e.g., skip connections 331, 332, 333, 334) between one or more layers of the encoder network 310 and the decoder network 320. The skip connections (e.g., skip connections 331, 332, 333, 334) between encoder blocks and decoder blocks add the feature maps to the symmetric transposed convolution blocks. The skip connections (e.g., skip connections 331, 332, 333, 334) may transfer the signal details from the encoder network 310 to the decoder network 320, which may aid the recovery of the clean signal. For example, the skip connections (e.g., skip connections 331, 332, 333, 334) may be used to overcome the optimization difficulty caused by the vanishing gradient phenomena in deep learning architecture.

The mean squared error (MSE) may be used between the uncorrupted and the reconstructed signals, with L1 regularization on the model parameters as the loss criterion of the DCAE network 300. For example, this may prevent possible over-smoothing due to the MSE loss function. The loss function may be represented as J=Σᵢ₌₁^{N}|xᵢ−ŷᵢ|₂²+λ|w|₁. J may represent the loss criterion, which consists of MSE loss between the uncorrupted and the reconstructed signals with L1 regularization added by the parameter λ. W may represent the DCA model 300 parameters and xᵢ and ŷᵢ may represent the original and the reconstructed signal for the i^{th} example. N may represent the total number of training samples.

One or more training data sets may be used to train the DCAE network 300. As an example, a training data set may include physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions. As an example, a training data set may include physiological signals associated with a prevalence of artifacts. As an example, a training data set may include physiological signals associated with one or more subjects experiencing central nervous system oxygen toxicity (CNS-OT) conditions. As an example, a training data set may include physiological signals associated with a first sub data set that may include physiological signals associated with a reduction of artifacts collected from both hands of one or more subjects and a second sub data set that may include physiological signals associated with a prevalence of artifacts collected from a first hand of one or more subjects and physiological signals associated with a reduction of artifacts collected from a second hand of one or more subjects.

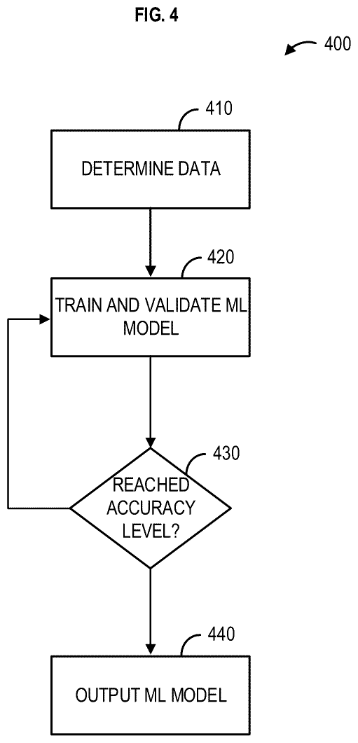

FIG. 4 shows a flowchart of an example training method 400 for generating the machine learning model (e.g., deep convolutional autoencoder (DCAE) network). The training 400 may be implemented by one or more user devices (e.g., the device 101, the electronic device 104, and/or the server 106). At step 410, one or more training data sets associated with one or more subjects may be determined (e.g., access, receive, retrieve, etc.). As an example, a training data set may include physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions. As an example, a training data set may include physiological signals associated with a prevalence of artifacts. As an example, a training data set may include physiological signals associated with one or more subjects experiencing central nervous system oxygen toxicity (CNS-OT) conditions. As an example, a training data set may include physiological signals associated with a first sub data set that may include physiological signals associated with a reduction of artifacts collected from both hands of one or more subjects and a second sub data set that may include physiological signals associated with a prevalence of artifacts collected from a first hand of one or more subjects and physiological signals associated with a reduction of artifacts collected from a second hand of one or more subjects.

At step 420, the machine learning model (e.g., DCAE network) may be trained based on the one or more training data sets. In an example, a first portion of the training data sets may be used to train the machine learning model (e.g., DCAE network). As an example, the DCAE may be trained and optimized by using an Adam optimizer with an initial learning rate of 0.001. The learning rate may be reduced by a factor of 0.1 after every five epochs. The training may end after 20 epochs.

At step 430, the machine learning model (e.g., DCAE network) may be evaluated using a second portion of the training data sets. For example, the machine learning model (e.g., DCAE network) may be evaluated to determine whether the predicted values have achieved a desired accuracy level. Once the desired accuracy level is achieved the machine learning model (e.g., DCAE network) may be output at step 440.

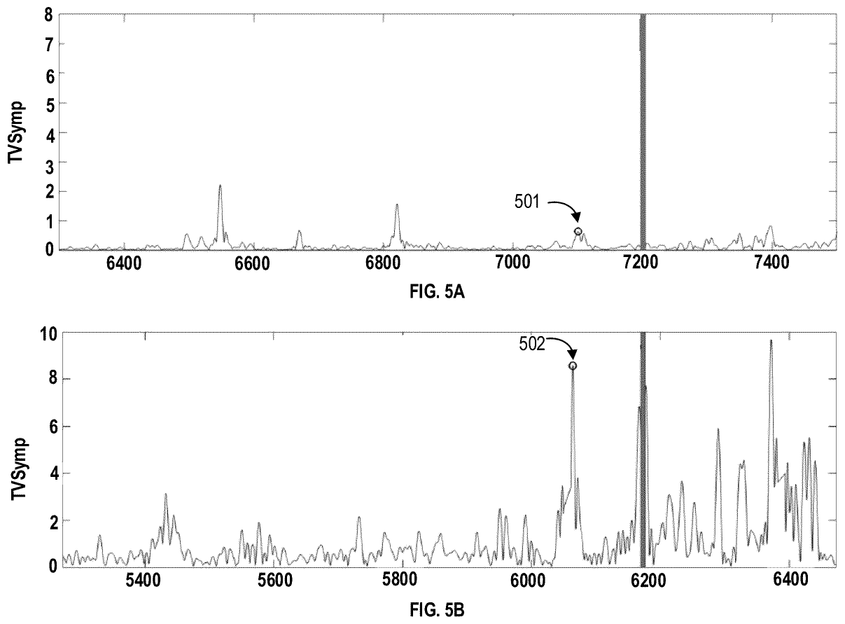

FIGS. 5A-5B show example physiological measurements (e.g., TVSymp) associated with a subject. For example, physiological data (e.g., EDA signal) associated with a subject may be used to generate a physiological measurement. The physiological measurement may include a time-invariant and a time-variant spectral analysis of the EDA signal (TVSymp). In an example, to compute the TVSymp, the time-frequency representation of EDA may be computed using variable frequency complex demodulation (VFCDM), which may enable a more accurate amplitude estimation along and may obtain increased time-frequency resolutions. At a sampling frequency of VFCDM decomposition at 2 Hz, the second and third components, including the approximate frequency range 0.08-0.25 Hz, may be used to compute TVSymp. Amplitudes of the time-varying components in this band at each time point may be summed together to obtain an estimated reconstructed EDA signal, X′(t). X′(t) may be normalized to a unit variance (making TVSymp a dimensionless quantity), and its instantaneous amplitude may be computed using the Hilbert transform, such as

$${Y}^{′} ( t ) = \frac{1}{π} P {\int }_{- ∞}^{∞} {X}^{′} ( τ ) / ( t - τ ) d τ .$$

P may indicate the Cauchy principal value. X′(t) and Y′(t) may form the complex conjugate pair. Thus, an analytic signal, Z(t), may be defined as Z(t)=X′(t)+iY′(t)=a(t)e^{jθ(t)}, where a(t)=[X′²(t)+Y′²(t)]^{1/2}, and θ(t)=arctan(Y′(t)/X′ (t)). The resulting a(t) may be the instantaneous amplitude of Z(t) and may correspond to the TVSymp time series.

FIG. 5A shows an example physiological measurement (e.g., TVSymp) associated with a subject without CNS-OT exposure while FIG. 5B shows an example physiological measurement (e.g., TVSymp) associated with a subject exhibiting symptoms (e.g., diaphoresis) of CNS-OT. FIG. 5B shows a large increase in the physiological measurement (e.g., TVSymp) amplitude that may precede the occurrence of symptoms of CNS-OT. For example, as shown in FIG. 5B, the physiological measurement (e.g., TVSymp) may reach a value higher than 8, as shown by the circles 501, 502 in FIGS. 5A-5B. In an example, the physiological measurement (e.g., TVSymp) may show several prominent peaks even after the end of the HBO₂.

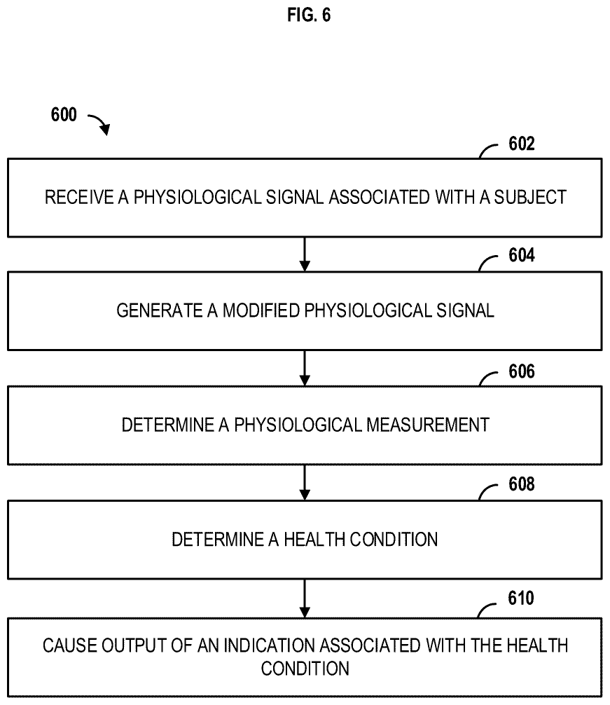

FIG. 6 shows a flowchart of an example method 600. Method 600 may be implemented by a user device (e.g., device 101, electronic device 104, sever 106). For example, the user device may include a smart watch, a smart phone, a laptop computer, a tablet computer, a desktop computer, a server, and the like. At step 602, a physiological signal associated with a subject may be received. For example, the physiological signal may be received from one or more sensors by the user device. The physiological signal may include an electrodermal activity (EDA) signal. The EDA signal may include artifacts. For example, the artifacts may be caused by one or more of motion noise or electronic noise. For example, motion artifacts may be caused by variable conduction of signals as the subject moves above and electronic noise may be caused by radiofrequency and magnetic interference.

At step 604, a modified physiological signal may be generated based on an application of a machine learning model to the physiological signal. For example, the user device may apply the machine learning model to the physiological signal to generate the modified physiological signal. The modified physiological signal may include the physiological signal with a reduction of the artifacts. The machine learning model may include a deep convolutional autoencoder (DCAE) network. The DCAE network may be trained based on one or more training data sets. As an example, a training data set may include physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions. As an example, a training data set may include physiological signals associated with a prevalence of artifacts. As an example, a training data set may include physiological signals associated with one or more subjects experiencing central nervous system oxygen toxicity (CNS-OT) conditions. As an example, a training data set may include physiological signals associated with a first sub data set that may include physiological signals associated with a reduction of artifacts collected from both hands of one or more subjects and a second sub data set that may include physiological signals associated with a prevalence of artifacts collected from a first hand of one or more subjects and physiological signals associated with a reduction of artifacts collected from a second hand of one or more subjects.

At step 606, a physiological measurement may be determined based on the modified physiological signal. For example, the user device may determine the physiological measurement based on the modified physiological signal. The physiological measurement may include a time-invariant and/or a time-variant spectral analysis of the EDA signal (TVSymp).

At step 608, a health condition may be determined based on a change in the physiological measurement satisfying a threshold. For example, the user device may determine the health condition based on the change in the physiological measurement satisfying a threshold. The change may be based on an increase in phasic components of the EDA signal. For example, the change may be caused by stress experienced by the subject based on breathing performed by the subject during prolonged exposure to HBO₂. The health condition may include one or more of a risk of seizure, a risk of CNS-OT, or symptoms of CNS-OT. In an example, it may be first determined that the physiological measurement satisfies a condition. The condition may include one or more of an autonomic induced elevation of a phasic component of the EDA signal or a non-autonomic induced elevation of a phasic component of the EDA signal. The change in the physiological measurement may be determined to satisfy the threshold based on the physiological measurement satisfying the condition.

At step 610, an indication associated with the health condition may be output. For example, the user device may output the health condition to the subject via a display (e.g., interface) of the user device. For example, the indication may include an alert notifying the user (e.g., subject) of a possible impending seizure or other health conditions such as a risk of CNS-OT, or that the user (e.g., subject) may be experiencing symptoms of CNS-OT.

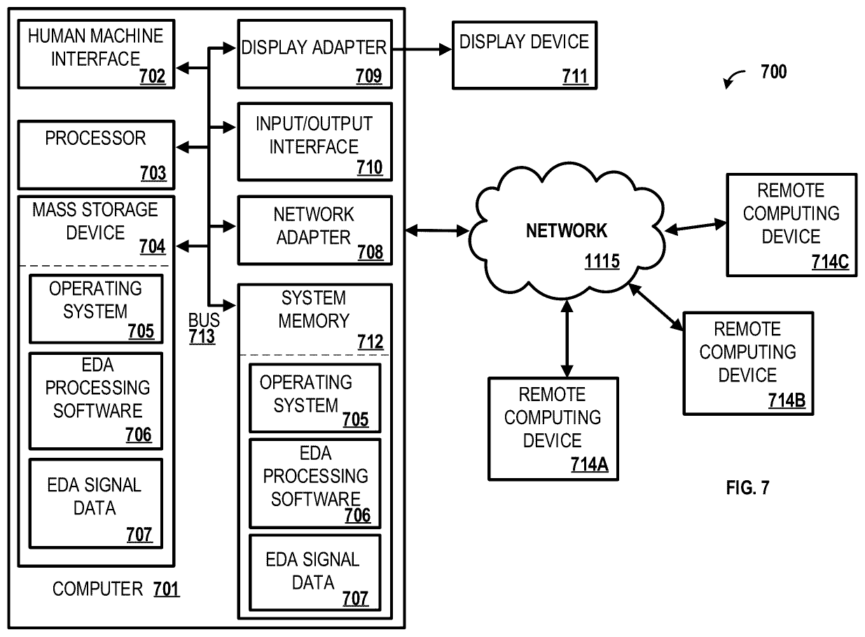

In an example, the methods and systems may be implemented on a computer 701 as shown in FIG. 7 and described below. By way of example, device 101, electronic device 104, and server 106 of FIG. 1 may be a computer 701 as shown in FIG. 7. Similarly, the methods and systems disclosed can utilize one or more computers to perform one or more functions in one or more locations. FIG. 7 shows a block diagram of an example operating environment 700 for performing the disclosed methods. For example, operating environment 700 is only an example of an operating environment and is not intended to suggest any limitation as to the scope of use or functionality of operating environment architecture. Neither should the operating environment 700 be interpreted as having any dependency or requirement relating to any one or combination of components illustrated in the example operating environment 700.

The present methods and systems can be operational with numerous other general purpose or special purpose computing system environments or configurations. Examples of well-known computing systems, environments, and/or configurations that can be suitable for use with the systems and methods include, but are not limited to, personal computers, server computers, laptop devices, and multiprocessor systems. Additional examples include set top boxes, programmable consumer electronics, network PCs, minicomputers, mainframe computers, distributed computing environments that include any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules include computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media such as memory storage devices.

Further, one skilled in the art will appreciate that the systems and methods disclosed herein can be implemented via a general-purpose computing device in the form of a computer 701. The computer 701 can include one or more components, such as one or more processors 703, a system memory 712, and a bus 713 that couples various components of the computer 701 comprising the one or more processors 703 to the system memory 712. In the case of multiple processors 703, the computer 701 may utilize parallel computing.

The bus 713 may include one or more of several possible types of bus structures, such as a memory bus, memory controller, a peripheral bus, an accelerated graphics port, or local bus using any of a variety of bus architectures. By way of example, such architectures can include an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 713, and all buses specified in this description can also be implemented over a wired or wireless network connection and one or more of the components of the computer 701, such as the one or more processors 703, a mass storage device 704, an operating system 705, EDA processing software 706, EDA signal data 707, a network adapter 708, the system memory 712, an Input/Output Interface 710, a display adapter 709, a display device 711, and a human machine interface 702, can be contained within one or more remote computing devices 714A-714C at physically separate locations, connected through buses of this form, in effect implementing a fully distributed system.

The computer 701 may operate on and/or include a variety of computer-readable media (e.g., non-transitory). Computer-readable media may be any available media that is accessible by the computer 701 and includes non-transitory, volatile, and/or non-volatile media, removable and non-removable media. The system memory 712 has computer-readable media in the form of volatile memory, such as random access memory (RAM), and/or non-volatile memory, such as read-only memory (ROM). The system memory 712 typically can include data such as the EDA signal data 707 and/or program modules such as the operating system 705 and the EDA processing software 706 that are accessible to and/or are operated on by the one or more processors 703.

The computer 701 may also include other removable/non-removable, volatile/non-volatile computer storage media. The mass storage device 704 may provide non-volatile storage of computer code, computer-readable instructions, data structures, program modules, and other data for the computer 701. The mass storage device 704 may be a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Any number of program modules can be stored on the mass storage device 704, such as, by way of example, the operating system 705 and the EDA processing software 706. One or more of the operating system 705 and the EDA processing software 706 (or some combination thereof) can include elements of the programming and the EDA processing software 706. The EDA signal data 707 can also be stored on the mass storage device 704. The EDA signal data 707 can be stored in any of one or more databases known in the art. Examples of such databases include, DB2®, Microsoft® Access, Microsoft® SQL Server, Oracle®, mySQL, PostgreSQL, and the like. The databases can be centralized or distributed across multiple locations within the network 715.

A user may enter commands and information into the computer 701 via an input device (not shown). Such input devices may include, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 703 via a human-machine interface 702 that is coupled to the bus 713, but may be connected by other interface and bus structures, such as a parallel port, game port, an IEEE 1394 Port (also known as a Firewire port), a serial port, network adapter 708, and/or a universal serial bus (USB).

A display device 711 may also be connected to the bus 713 via an interface, such as a display adapter 709. It is contemplated that the computer 701 may have more than one display adapter 709 and the computer 701 may have more than one display device 711. A display device 711 may be a monitor, an LCD (Liquid Crystal Display), light-emitting diode (LED) display, television, smart lens, smart glass, and/or a projector. In addition to the display device 711, other output peripheral devices may include components such as speakers (not shown) and a printer (not shown) which may be connected to the computer 701 via Input/Output Interface 710. Any step and/or result of the methods may be output (or caused to be output) in any form to an output device. Such output may be any form of visual representation, including, but not limited to, textual, graphical, animation, audio, tactile, and the like. The display 711 and computer 701 may be part of one device, or separate devices.

The computer 701 can operate in a networked environment using logical connections to one or more remote computing devices 714A-714C. By way of example, a remote computing device 714A-714C can be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computer 701 and a remote computing device 714A-714C can be made via a network 715, such as a local area network (LAN) and/or a general wide area network (WAN). Such network connections can be through the network adapter 708. The network adapter 708 can be implemented in both wired and wireless environments. Such networking environments are conventional and commonplace in dwellings, offices, enterprise-wide computer networks, intranets, and the Internet.

Application programs and other executable program components such as the operating system 705 are illustrated herein as discrete blocks, although it is recognized that such programs and components can reside at various times in different storage components of the computing device 701, and are executed by the one or more processors 703 of the computer 701. An implementation of the EDA processing software 706 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can include “computer storage media” and “communications media.” “Computer storage media” can include volatile and non-volatile, removable and non-removable media implemented in any methods or technology for storage of information such as computer readable instructions, data structures, program modules, or other data. For example, computer storage media may include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

Each of the elements described in the present disclosure may include one or more components, and names thereof may vary depending on a type of an electronic device. The electronic device according to various exemplary embodiments may include at least one of the elements described in the present disclosure. Some of the elements described herein may be omitted and/or additional other elements may be further included. Further, some of the elements of the electronic device, according to various exemplary embodiments, may be combined and constructed as a single entity, so as to equally perform functions of the corresponding elements before combination.

It is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise.

Throughout the description and claims of this specification, the words “include” and/or “comprise” and variations of the words, such as “including,” “includes,” “comprising,” and “comprises,” mean “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components (e.g., apparatuses) that can be used to perform the disclosed methods and can be part of the disclosed systems. These and other components are disclosed herein, and it is understood that when combinations, subsets, interactions, groups, etc. of these components are disclosed that while specific reference of each various individual and collective combinations and permutations of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium (e.g., non-transitory) having processor-executable instructions (e.g., computer software) embodied in the storage medium. More particularly, the present methods and systems may take the form of web-implemented computer software. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Some additional examples of machine learning concepts and techniques that may be useful with the teachings herein include, for example, Supervised Machine Learning models, such as: “Logistic Regression” which may be used to determine if an input belongs to a certain group or not; “Support Vector Machines” which may be used to create coordinates for each object in an n-dimensional space and uses a hyperplane to group objects by common features; “Naive Bayes” which is an algorithm that may assume independence among variables and use probability to classify objects based on features; “Decision trees” which are also classifiers that may be used to determine what category an input falls into by traversing the leafs and nodes of a tree; “Linear Regression” which may be used to identify relationships between the variable of interest and the inputs, and predict its values based on the values of the input variables; “k Nearest Neighbors” technique which involves grouping the closest objects in a dataset and finding the most frequent or average characteristics among the objects; “Random Forest” which is a collection of many decision trees from random subsets of the data, resulting in a combination of trees that may be more accurate in prediction than a single decision tree; “Boosting Algorithms”, such as Gradient Boosting Machine, XGBoost, and LightGBM, which use ensemble learning to combine predictions from multiple algorithms (such as decision trees) while taking into account the error from the previous algorithm. Additionally, Unsupervised Machine Learning models such as “K-Means” which is an algorithm that finds similarities between objects and groups them into K different clusters; and “Hierarchical Clustering” which build a tree of nested clusters without having to specify the number of clusters, can provide some functionality.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. An apparatus for predicting a health condition of a subject comprising:
one or more processors; and
a memory storing processor-executable instructions that, when executed by the one or more processors, cause the apparatus to:
receive a physiological signal associated with the subject, wherein the physiological signal comprises artifacts;
generate, based on an application of a machine learning model to the physiological signal, a modified physiological signal, wherein the modified physiological signal comprises the physiological signal with a reduction of the artifacts;
determine, based on the modified physiological signal, a physiological measurement;
determine, based on a change in the physiological measurement satisfying a threshold, the health condition; and
cause output of an indication associated with the health condition.

2. The apparatus of claim 1, wherein the physiological signal comprises an electrodermal activity (EDA) signal, and wherein the physiological measurement comprises a time-invariant or a time-variant spectral analysis of the EDA signal (TVSymp).

3. The apparatus of claim 2, wherein the change is based on an increase in phasic components of the EDA signal, and wherein the change is caused by stress based on breathing performed by the subject during prolonged exposure to hyperbaric oxygen (HBO₂).

4. The apparatus of claim 2, wherein the processor-executable instructions that, when executed by the one or more processors, cause the apparatus to determine, based on the change in the physiological measurement satisfying the threshold, the health condition, further cause the apparatus to:
determine, based on the physiological measurement satisfying a condition, the change in the physiological measurement satisfies the threshold, wherein the condition comprise an autonomic induced elevation of a phasic component of the EDA signal or a non-autonomic induced elevation of a phasic component of the EDA signal; and
determine, based on the change in the physiological measurement satisfying the threshold, the health condition.

5. The apparatus of claim 1, wherein the machine learning model comprises a deep convolutional autoencoder network.

6. The apparatus of claim 1, wherein the machine learning model is trained based on one or more training data sets.

7. The apparatus of claim 6, wherein the one or more training data sets comprise one or more of:
a first training data set comprising physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions,
a second training data set comprising physiological signals associated with a prevalence of artifacts,
a third training data set comprising physiological signals associated with one or more subjects experiencing central nervous system oxygen toxicity (CNS-OT) conditions, and
a fourth training data set comprising physiological signals associated with a first sub data set comprising physiological signals associated with a reduction of artifacts collected from both hands of one or more subjects and a second sub data set comprising physiological signals associated with a prevalence of artifacts collected from a first hand of one or more subjects and physiological signals associated with a reduction of artifacts collected from a second hand of one or more subjects.

8. The apparatus of claim 1, wherein the health condition comprises one or more of a risk of seizure, a risk of CNS-OT, or symptoms of CNS-OT.

9. One or more non-transitory computer-readable media storing processor-executable instructions that, when executed by at least one processor, cause the at least one processor to:
receive, by a computing device, a physiological signal associated with a subject, wherein the physiological signal comprises artifacts;
generate, based on an application of a machine learning model to the physiological signal, a modified physiological signal, wherein the modified physiological signal comprises the physiological signal with a reduction of the artifacts;
determine, based on the modified physiological signal, a physiological measurement;
determine, based on a change in the physiological measurement satisfying a threshold, a health condition; and
cause an output of an indication of the health condition.

10. The non-transitory computer-readable media of claim 9, wherein the physiological signal comprises an electrodermal activity (EDA) signal, and wherein the physiological measurement comprises a time-invariant or a time-variant spectral analysis of the EDA signal (TVSymp).

11. The non-transitory computer-readable media of claim 10, wherein the change is based on an increase in phasic components of the EDA signal, and wherein the change is caused by stress based on breathing performed by the subject during prolonged exposure to hyperbaric oxygen (HBO₂).

12. The non-transitory computer-readable media of claim 10, wherein the processor-executable instructions that, when executed by the at least one processor, cause the at least one processor to determine, based on the change in the physiological measurement satisfying the threshold, the health condition, further cause the at least one processor to:
determine, based on the physiological measurement satisfying a condition, the change in the physiological measurement satisfies the threshold, wherein the condition comprise an autonomic induced elevation of a phasic component of the EDA signal or a non-autonomic induced elevation of a phasic component of the EDA signal; and
determine, based on the change in the physiological measurement satisfying the threshold, the health condition.

13. The non-transitory computer-readable media of claim 9, wherein the machine learning model comprises a deep convolutional autoencoder network.

14. The non-transitory computer-readable media of claim 9, wherein the machine learning model is trained based on one or more training data sets.

15. The non-transitory computer-readable media of claim 14, wherein the one or more training data sets comprise one or more of:
a first training data set comprising physiological signals from a public data set associated with physiological stimuli based on one or more of an auditory task, pain induced by electrical stimulation, a visual detection task, fear conditioning tasks, being shown aversive or neutral pictures, being shown pictures while subjected to auditory distractors, or being shown pictures of facial expressions,
a second training data set comprising physiological signals associated with a prevalence of artifacts,
a third training data set comprising physiological signals associated with one or more subjects experiencing central nervous system oxygen toxicity (CNS-OT) conditions, and
a fourth training data set comprising physiological signals associated with a first sub data set comprising physiological signals associated with a reduction of artifacts collected from both hands of one or more subjects and a second sub data set comprising physiological signals associated with a prevalence of artifacts collected from a first hand of one or more subjects and physiological signals associated with a reduction of artifacts collected from a second hand of one or more subjects.

16. The non-transitory computer-readable media of claim 9, wherein the health condition comprises one or more of a risk of seizure, a risk of CNS-OT, or symptoms of CNS-OT.

17. A system for predicting a health condition of a subject associated with prolonged exposure to hyperbaric oxygen (HBO₂) comprising:
a sensor configured to be affixed to a surface of skin of the subject, wherein the sensor is configured to receive a physiological signal associated with the subject based on measuring a conductance associated with the surface of skin of the subject;
a display configured to output an interface to the subject; and
a computing device in communication with the sensor and the display, wherein the computing device is configured to:
receive, from the sensor, the physiological signal, wherein the physiological signal comprises artifacts;
provide the physiological signal to a machine learning model;
generate, based on the machine learning model, a modified physiological signal, wherein the modified physiological signal comprises the physiological signal with a reduction of the artifacts;
determine, based on the modified physiological signal, a physiological measurement;
determine a change in the physiological measurement satisfies a threshold, wherein the change is caused by stress based on breathing performed by the subject during prolonged exposure to HBO₂;
determine, based on the change in the physiological measurement satisfying the threshold, the health condition; and
cause the display to output an indication of the health condition via the interface to the subject.

18. The system of claim 17, wherein the physiological signal comprises an electrodermal activity (EDA) signal, and wherein the physiological measurement comprises a time-invariant or a time-variant spectral analysis of the EDA signal (TVSymp).

19. The system of claim 17, wherein the machine learning model comprises a deep convolutional autoencoder network.

20. The system of claim 17, wherein the health condition comprises one or more of a risk of seizure, a risk of CNS-OT, or symptoms of CNS-OT.
